(19) United States
(12) Patent Application Publication
(10) Pub. No.: US 2019/0112272 A1
(43) Pub. Date: Apr. 18, 2019

(54) SYNTHETIC RETINOIDS FOR USE IN RAR MEDIATED CONDITIONS

(71) Applicants: High Force Research Limited, Durham (GB); University of Durham, Durham (GB); The University Court of The University of Aberdeen, Aberdeen (GB)

(72) Inventors: Andrew Whiting, Durham (GB); Roy Valentine, Durham (GB); David Chisholm, Durham (GB); Peter McCaffery, Aberdeen (GB); Iain Greig, Aberdeen (GB); Thabat Khatib, Aberdeen (GB)

(21) Appl. No.: 16/092,106

(22) PCT Filed: Apr. 6, 2017
(86) PCT No.: PCT/GB2017/050977

§ 371 Date: Oct. 8, 2018

(30) Foreign Application Priority Data
Apr. 7, 2016 (GB) 1605913.1

(51) Int. Cl. C07D 215/06; C07D 209/08; C07D 417/04; C07C 63/66; A61P 25/16; A61P 25/08; A61P 25/28

(52) U.S. Cl. CPC C07D 215/06; C07D 209/08; C07D 417/04; C07C 2602/10; A61P 25/16; A61P 25/08; A61P 25/28; C07C 63/66

(57) ABSTRACT

There are described novel compounds of formula I: in which R¹, R², R³, R⁴, R⁵, X¹ and X² are each as herein defined, for use in the treatment or alleviation of an RAR miated condition: and methods related thereto.

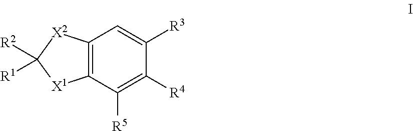

### FIELD OF THE INVENTION

The present invention relates to the treatment of RAR mediated conditions, to novel compounds, their use and methods of treatment related thereto.

More particularly, the present invention relates to the use of synthetic retinoid compounds in the treatment or alleviation of RAR mediated conditions, such as, neurodegenerative disorders. Certain of the synthetic retinoid compounds are novel per se, therefore the present invention also relates to such novel compounds and methods of their preparation.

### BACKGROUND TO THE INVENTION

Vitamin A (retinol) and its derivatives belong to a class of compounds known as retinoids. Retinoids are an important class of signalling molecules that are involved in controlling many important biological pathways from embryogenesis through to adult homeostasis and many aspects of stem cell development, such as, stem cell proliferation, differentiation and apoptosis.

Retinoids are structurally and/or functionally related to vitamin A; and many possess biological activity including all-trans-retinoic acid (ATRA). ATRA is the most biologically active endogenous retinoid and has been widely studied for many years; ATRA isomerises under physiological and experimental conditions, with different isomers activating different receptors, which may contribute to the variety of biological effects observed with these small molecules.

Neurodegenerative conditions can seriously affect a person's ability to carry out normal daily activities. Such neurodegenerative disorders include multiple sclerosis, dementia, Alzheimer's disease, Parkinson's disease, stroke, traumatic brain injury, ALS (amyotrophic lateral sclerosis) and the like.

According to the Alzheimer's Society there are currently 800,000 people with dementia in the UK, and it is estimated that by 2021 there will be over a million people with dementia in the UK.

In the UK 60,000 deaths a year are directly attributable to dementia and it is estimated that by delaying the onset of dementia by 5 years deaths directly attributable to dementia would be reduced by 30,000 a year.

Furthermore, 80% of people living in care homes have a form of dementia or severe memory problems. The financial cost of dementia to the UK was estimated to be in excess of £23 billion in 2012.

Alzheimer's disease is also associated with brain-related neurodegeneration, with progressive cognitive decline, functional impairment and loss of independence. The condition places enormous financial and psychological burdens on Caregivers and Healthcare Providers. Worldwide prevalence has been estimated as being in the region of 35 million in 2010, with levels projected to increase as life expectancy increases in the developed world.

The number of deaths caused by Alzheimer's disease is generally thought to be underreported, but a recent study has identified Alzheimer's disease as the potential third-leading cause of death in the USA after heart disease and cancer.

There is a stark unmet medical need for a therapy for the treatment Alzheimer's disease. Current therapies include NMDA antagonists and cholinesterase inhibitors, but they can provide only brief symptomatic relief. A minority of patients see a small cognitive improvement and a majority see a brief (up to 12 months) delay in cognitive decline. The disease is expected to affect over 100 million people worldwide by 2050 and is estimated to lead to annual costs of well over $600 billion. A major cost is that associated with the often-ignored relatives who act as carers of those afflicted with the disease. Whilst a drug that can cure or prevent Alzheimer's disease represents the ultimate goal of most development programmes, a more realistic aim is to develop drugs which could delay the onset or slow the development of the disease, either of which would have enormous socioeconomic implications by reducing the number of years of care required by patients and carers.

The lack of either effective current drugs or promising drugs in the late-stage pipeline is highlighted by the anticipated drop in the market for Alzheimer's disease drugs, in spite of the enormous costs associated with the disease, to a mere $4 billion by 2018, less than the value of a number of individual drugs for other diseases. As of 2013, there were 102 drugs in clinical trials for the treatment of Alzheimer's disease, but most of these target β-amyloid or neuromodulatory signalling. Results from clinical trials have been, at best, underwhelming: showing modest cognitive benefits in patients with mild to moderate disease, whilst relying on the ability to identify these patients in timely and cost-effective fashion, as to allow for treatment during the window of opportunity. Recent notable disappointments have included antibodies against β-amyloid (gantenerumab, solanezumab, bapineuzumab and aducanumab), γ-secretase inhibitors (semgacestat), β-secretase inhibitors (LY2886721) and the retinoid bexarotene.

Retinoic acid is an endogenous signalling molecule that regulates transcription by activating RAR nuclear receptors. Retinoic acid is a small lipophilic molecule that crosses tissue barriers and is useful as a drug to treat skin disorders as well as a variety of cancers.^{1,2} Retinoic acid is also potent in promoting neuronal survival and neurite outgrowth, suggesting potential as a therapy for improving cognition in dementia and other neurodegenerative disorders.

Furthermore, endogenous retinoic acid levels decline in the ageing human³ and rodent^{4,5} brain and its parent molecule, vitamin A also falls with age in humans.^{6,7} Because retinoic acid supports neuroplasticity essential for learning and memory,⁸ this decline in concentration weakens cognitive function. Boosting the retinoic acid signal with synthetic ligands for the RAR receptors improves cognition in Alzheimer's disease (AD) model mice, clearing Aβ in both neurons and microglia, as well as providing a strong anti-inflammatory action.⁹ In addition, vitamin A and retinoic acid are protective against Aβ neurotoxicity.^{10,11} Importantly, retinoic acid has an efficacious effect in multiple models of AD; it also improves cognition and provides anti-inflammatory action in a diabetic model of AD.¹² It is these multiple physiological actions of retinoic acid that make it a promising treatment for AD.

Therefore, there is a need for a novel treatment of neurodegenerative conditions, such as Alzheimer's disease.

International Patent application No. PCT/GB2007/003237 (WO 2008/025965) discloses retinoid compounds which exhibited good stability and induced cell differentiation.

Our unpublished International Patent application No. PCT/GB2015/052956 discloses novel tetrahydro- and dihydro-quinoline retinoid compounds that are useful as fluorescent probes.

### SUMMARY OF THE INVENTION

Thus, the present invention provides compounds which, inter alia, are selective retinoic acid receptor (RAR) agonists. The present invention also pertains to pharmaceutical compositions comprising such compounds, and the use of such compounds and compositions, both in vitro and in vivo, to (selectively) activate RAR, and in the treatment of diseases and conditions that are mediated by RAR and may be therefore be alleviated by the activation of RAR.

Such RAR mediated conditions include neurodegenerative disorders, including cognitive disorders, memory impairment, memory deficit, senile dementia, Alzheimer's disease, early stage Alzheimer's disease, intermediate stage Alzheimer's disease, late stage Alzheimer's disease, cognitive impairment, mild cognitive impairment and ALS (amyotrophic lateral sclerosis).

The present invention provides the use of EC23® type molecules in the treatment for neurodegenerative disorders, such as Alzheimer's disease.

Thus, according to a first aspect of the invention there is provided a compound of formula I:

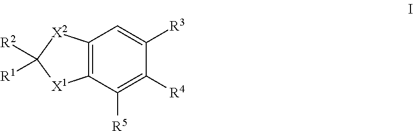

in which
X¹ is —NCHR⁶R⁷, —CR⁸R⁹;
X² is —CR¹⁰R¹¹—CR¹²R¹⁴ or —CR¹⁵R¹⁶;
R¹, R², R¹⁰ and R¹¹, which may be the same or different, are each hydrogen or alkyl C1-4, or together one pair of R¹ and R¹⁰ or R² and R¹¹ represent a 5- or 6-membered ring, or together one pair of R¹ and R¹⁰ or R² and R¹¹ represent a bond;
R⁴ and R⁵ are each hydrogen;
R⁶, R⁷, R⁸ and R⁹, which may be the same or different, are each hydrogen, alkyl C1-10 acyl;
R¹² and R¹⁴, which may be the same or different, are each hydrogen, alkyl C1-4; or together one pair of R¹⁰ and R¹² or R¹¹ and R¹⁴ represent a bond, or R¹² and R¹⁴ together form a group:

═CR¹⁷R¹⁸
provided that the pair of R¹⁰ and R¹² or R¹¹ and R¹⁴ does not represent a bond if a pair from R¹, R², R¹⁰ and R¹¹ represents a bond;
R¹⁵ and R¹⁶, which may be the same or different, are each hydrogen or alkyl C1-4, or together one pair of R¹ and R¹⁵ or R² and R¹⁶ represent a 5- or 6-membered ring;
R¹⁷ and R¹⁸, which may be the same or different, are each hydrogen or alkyl C1-10; and
R³ is a group II, III, IV, V, VI, VII, X, XI or XII:

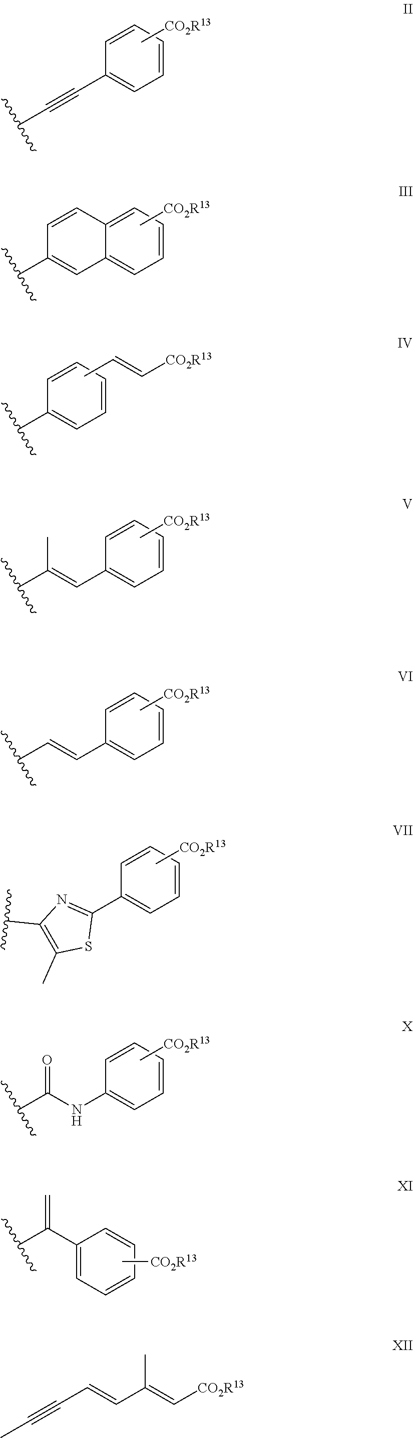

in which R¹³ is h_{y}drogen, alkyl C1-10 or haloalkyl C1-10;
and isomers thereof;
in free or in salt form;
for use n the treatment or alleviation of an RAR mediated condition.

As used herein, the term “alkyl” refers to a fully saturated, branched, unbranched or cyclic hydrocarbon moiety, i.e. primary, secondary or tertiary alkyl or, where appropriate, cycloalkyl or alkyl substituted by cycloalkyl, they may also be saturated or unsaturated alkyl groups. Where not otherwise identified, preferably the alkyl comprises 1 to 10 carbon atoms, more preferably 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tent-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein the term “aryl” refers to an aromatic monocyclic or multicyclic hydrocarbon ring system consisting only of hydrogen and carbon and containing from 6 to 19 carbon atoms, preferably 6 to 10 carbon atoms, where the ring system may be partially saturated. Aryl groups include, but are not limited to groups such as fluorenyl, phenyl, indenyl and naphthyl. Unless stated otherwise specifically in the specification, the term “aryl” or the prefix “ar-” (such as in “aralkyl”) is meant to include aryl radicals optionally substituted by one or more substituents selected from the group consisting of alkyl, alkenyl, alkynyl, halo, haloalkyl, cyano, nitro, amino, amidine, aryl, aralkyl, cycloalkyl, cycloalkylalkyl, heterocyclyl, heterocyclylalkyl, heteroaryl or heteroarylalkyl. Preferred aryl groups are optionally substituted phenyl or naphthyl groups.

An aryl group may be mono-, bi-, tri-, or polycyclic, preferably mono-, bi-, or tricyclic, more preferably mono- or bicyclic.

In one aspect of the invention R³ is a group II, III or IV as herein defined.

In one aspect of the invention R¹ is alkyl C1-10, preferably alkyl C1-3.

In one aspect of the invention X¹ is —NCHR⁶R⁷.

In one aspect of the invention X¹ is —CR⁸R⁹.

In one aspect of the invention X² is —CR¹⁰R¹¹—CR¹²R¹⁴.

In one aspect of the invention X² is —CR¹⁵R¹⁶.

In one aspect of the invention R¹, R², R¹⁰, R¹¹, R¹², R¹⁴, R¹⁵ and R¹⁶ are each hydrogen.

In one aspect of the invention one pair of R¹ and R¹⁰ or R² and R¹¹ represent a bond.

In one aspect of the invention R¹² and R¹⁴ are the same or different; R¹² and R¹⁴ may each represent alkyl C1-4, e.g. methyl.

As used herein, the term “halogen” or “halo” refers to fluoro, chloro, bromo, and iodo.

In another aspect of the invention R³ is a group II, as herein defined.

In another aspect of the invention R³ is a group III, as herein defined.

In another aspect of the invention R³ is a group IV, as herein defined.

In another aspect of the invention R³ is a group V, as herein defined.

In another aspect of the invention R³ is a group VI, as herein defined.

In another aspect of the invention R³ is a group VII, as herein defined.

In another aspect of the invention R³ is a group X, as herein defined.

In another aspect of the invention R³ is a group XI, as herein defined.

In another aspect of the invention R³ is a group XII, as herein defined.

The moiety —CO₂R¹³ is preferably in the 4-position, i.e. in the para position to the ethynyl group. Preferably R¹³ is hydrogen.

In one aspect of the invention there is provided a compound of formula I in which:
X² is —CR¹⁰R¹¹—CCR¹²R¹⁴; and
X¹, R¹, R², R³, R⁴ and R⁵ are each as herein defined;
and isomers thereof;
in free or in salt form;
for use in the treatmentor alleviation of an RAR mediated condition.

Such compounds of formula I in which X² is —CR¹⁰R¹¹—CR¹²R¹⁴; can generally be represented as compounds of formula XIII:

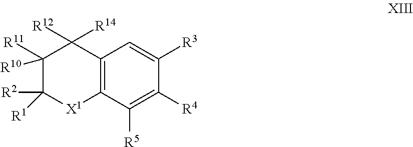

in which
X¹, R¹, R², R³, R⁴, R⁵, R¹⁰, R¹¹, R¹² _{and R}¹⁴, are each as herein defined.

In another aspect of the invention there is provided a compound of formula XIII:

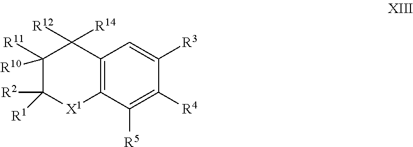

in which
X¹ is —CR⁸R⁹; and
R¹, R², R³, R⁴, R⁵, R⁸, R⁹, R¹⁰, R¹¹, R¹² and R¹⁴, are each as herein defined.
and isomers thereof;
in free or in salt form;
for use in the treatment or alleviation of an RAR mediated condition.

In another aspect of the invention there is provided a compound of formula XIII:

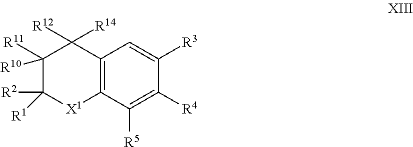

in which
X¹ is —NCHR⁶R⁷; and
and R¹, R², R³, R⁴, R⁵, R⁶, R⁷, R¹⁰, R¹¹, R¹² and R¹⁴, are each as herein defined.
and isomers thereof;
in free or in salt form;
for use in the treatment or alleviation of an RAR mediated condition.

In one aspect of the invention there is provided a compound of formula I in which:
X² is —CR¹⁵R¹⁶;
together R¹ and R¹⁵ represent a 6-membered ring; and
X¹, R², R³, R⁴ and R⁵ are each as herein defined.
and isomers thereof;
in free or in salt form;
for use in the treatment or alleviation of an RAR mediated condition.

Such compounds of formula I in which X² is —CR¹⁵R¹⁶ and together R¹ and R¹⁵ represent a 6-membered ring can generally be represented as compounds of formula XIV:

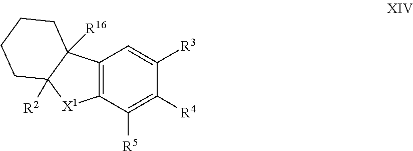

in which
X¹, R², R³, R⁴, R⁵ and R¹⁶ are each as herein defined;

and isomers thereof;
in free or in salt form;
for use in he treatment or alleviation of an RAR mediated condition.

In one aspect of the invention there is provided a compound of formula XIV:

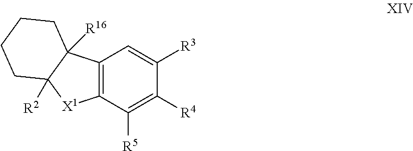

in which
X¹ is —NCHR⁶R⁷; and
R², R³, R⁴, R⁵, R⁶, R⁷ and R¹⁶ are each as herein defined;
and isomers thereof;
in free or in salt form;
for use in the treatment or alleviation of an RAR mediated condition.

In one aspect of the invention there is provided a compound of formula XIV:

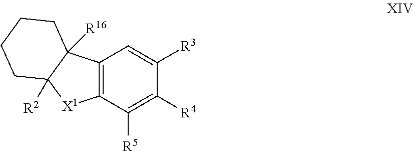

X¹ is —CR⁸R⁹; and
R², R³, R⁴, R⁵, R⁸, R⁹ and R¹⁶ are each as herein defined;
and isomers thereof;
in free or in salt form;
for use n the treatment or alleviation of an RAR mediated condition.

Specific compounds of formula I which may be mentioned include those selected from the group consisting of:
4-(5,5,8,8-tetramethyl-5,6,7,8-tetrahydro-naphthalen-2-ylethynyl) benzoic acid (EC23®);
4-2-[4,4-dimethyl-1-(propan-2-yl)-1,2,3,4-tetrahydroquinolin-6-yl]ethynylbenzoic acid (compound 9 of PCT/GB2015/052956);
6-(1,4,4-trimethyl-1,2,3,4-tetrahydroquinolin-6-yl)-naphthalene-2-carboxylic acid methyl ester (compound 11 of PCT/GB2015/052956);
3-[4-(1,4,4-trimethyl-1,2,3,4-tetrahydroquinolin-6-yl)-phenyl]-acrylic acid methyl ester (compound 13 of PCT/GB2015/052956); and
4-2-[2,4,4-trimethyl-1-(propan-2-yl)-1,4-dihydroquinolin-6-yl]ethynylbenzoic acid, (compound 17 of PCT/GB2015/052956);
in free or in salt form;
for use in the treatmentor alleviation of an RAR mediated condition.

An RAR mediated condition includes, but shall not be limited to, neurodegenerative conditions, for example, multiple sclerosis, Parkinson's disease, stroke, traumatic brain injury, epilepsy, cognitive disorders, memory impairment, memory deficit, senile dementia, Alzheimer's disease, early stage Alzheimer's disease, intermediate stage Alzheimer's disease, late stage Alzheimer's disease, cognitive impairment, mild cognitive impairment and ALS (amyotrophic lateral sclerosis). In a particular aspect of the present invention the RAR mediated condition is Alzheimer's disease. In another aspect of the present invention the RAR mediated condition is ALS (amyotrophic lateral sclerosis).

In another aspect of the present invention pertains to use of a compound as described herein, in the manufacture of a medicament for use in the treatment or alleviation of an RAR mediated condition as herein defined. In one embodiment, the medicament comprises the compound of formula I herein.

According to another aspect of the invention there is provided a method of treatment of a patient suffering an RAR mediated condition as herein defined, said method comprising administering to a patient a therapeutically effective amount of a compound of formula I herein.

According to another aspect of the invention there is provided a pharmaceutical composition comprising a compound of formula I as described herein, and a pharmaceutically acceptable adjuvant, diluent or carrier, for use in the treatment or alleviation of an RAR mediated condition as herein defined.

Another aspect of the invention provides a method of preparing a pharmaceutical composition as herein described, said method comprising the step of admixing a compound of formula I herein, and a pharmaceutically acceptable adjuvant, diluent or carrier.

Certain compounds of formula I are novel per se. Therefore, according to a further aspect of the invention there is provided a compound of formula I in which X² is —CR¹⁵R¹⁶ are novel per se.

Therefore, according to a further aspect of the invention there is provided a compound of formula I:

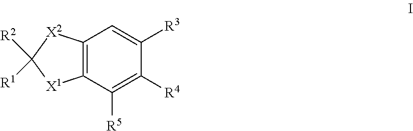

in which
X² is —CR¹⁵R¹⁶; and
X¹, R¹, R², R³, R⁴, R⁵, R¹⁵ and R¹⁶ are each as herein defined;
and isomers thereof;
in free or in salt form.

According to this aspect of the invention there is also provided compounds of formula I in which X² is —CR¹⁵R¹⁶ and together le and R¹⁵ represent a 6-membered ring, which can generally be represented as compounds of formula XIV:

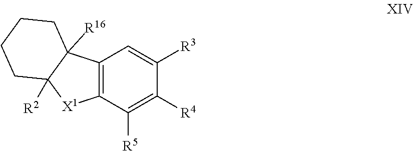

in which
X¹, R², R³, R⁴, R⁵ and R¹⁶ are each as herein defined;
and isomers thereof;
in free or in salt form.

In one aspect of the invention there is provided a compound of formula XIV:

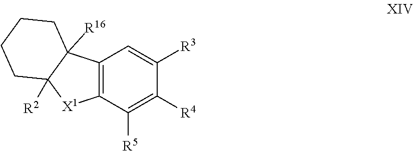

in which
X¹ is —NCHR⁶R⁷; and
R², R³, R⁴, R⁵, R⁶, R⁷ and R¹⁶ are each as herein defined;
and isomers thereof;
in free or in salt form.

In another aspect of the invention there is provided a compound of formula XIV:

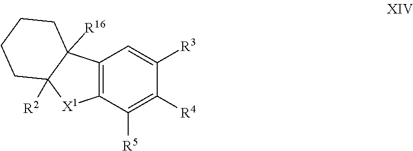

X¹ is —CR⁸R⁹; and
R², R³, R⁴, R⁵, R⁸, R⁹ and R¹⁶ are each as herein defined;
and isomers thereof;
in free or in salt form.

“Pharmaceutical composition” (or “pharmaceutically acceptable composition”) means a composition suitable for administration to a patient. Such compositions may contain the neat compound (or compounds) of the invention or mixtures thereof, or salts, solvates, prodrugs, isomers, or tautomers thereof, or they may contain one or more pharmaceutically acceptable carriers or diluents. The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like.

Those skilled in the art will recognize those instances in which the compounds of the invention may be converted to prodrugs and/or solvates, another embodiment of the present invention. A discussion of prodrugs is provided in T. Higuchi and V. Stella,

Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

One or more compounds of the invention may exist in unsolvated as well as solvated forms with pharmaceutically acceptable solvents such as water, ethanol, and the like, and it is intended that the invention embrace both solvated and unsolvated forms where they exist. “Solvate” means a physical association of a compound of the invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂0.

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

Those skilled in the art will recognize those instances in which the compounds of the invention may form salts. In such instances, another embodiment provides pharmaceutically acceptable salts of the compounds of the invention. Thus, reference to a compound of the invention herein is understood to include reference to salts thereof, unless otherwise indicated. The term “salt(s)”, as employed herein, denotes any of the following: acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also potentially useful. Salts of the compounds of the invention may be formed by methods known to those of ordinary skill in the art, for example, by reacting a compound of the invention with an amount of acid or base, such as an equivalent amount, in a medium such as one in which the salt precipitates or in an aqueous medium followed by lyophilisation.

Exemplary acid addition salts which may be useful include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartrates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered as potentially useful alternatives to the free forms of the corresponding compounds for purposes of the invention. Another embodiment which may be useful includes pharmaceutically acceptable esters of the compounds of the invention. Such esters may include the following groups:
(1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C1-4 alkyl, or C1-4 alkoxy or amino);
(2) sulfonate esters, such as alkyl- or aralkylsulfonyl (for example, methane sulfonyl);
(3) amino acid esters (for example, L-valyl or L-isoleucyl);
(4) phosphonate esters; and
(5) mono-, di- or triphosphate esters. The phosphate esters may be further esterified by, for example, a C1-20 alcohol or reactive derivative thereof, or by a 2,3-di-(C6-24)acyl glycerol.

Polymorphic forms of the compounds of the invention, and of the salts, solvates, esters and prodrugs of the compounds of the invention, are intended to be included in the present invention.

Another embodiment provides suitable dosages and dosage forms of the compounds of the invention. Suitable doses for administering compounds of the invention to patients may readily be determined by those skilled in the art, e.g., by an attending physician, pharmacist, or other skilled worker, and may vary according to patient health, age, weight, frequency of administration, use with other active ingredients, and/or indication for which the compounds are administered. Doses may range from about 0.001 to 500 mg/kg of body weight/day of the compound of the invention. In one embodiment, the dosage is from about 0.01 to about 25 mg/kg of body weight/day of a compound of the invention, or a pharmaceutically acceptable salt or solvate of said compound. In another embodiment, the quantity of active compound in a unit dose of preparation may be varied or adjusted from about 1 mg to about 100 mg, preferably from about 1 mg to about 50 mg, more preferably from about 1 mg to about 25 mg, according to the particular application. In another embodiment, a typical recommended daily dosage regimen for oral administration can range from about 1 mg/day to about 500 mg/day, preferably 1 mg/day to 200 mg/day, in two to four divided doses.

When used in combination with one or more additional therapeutic agents, the compounds of this invention may be administered together or sequentially. When administered sequentially, compounds of the invention may be administered before or after the one or more additional therapeutic agents, as determined by those skilled in the art or patient preference. If formulated as a fixed dose, such combination products employ the compounds of this invention within the dosage range described herein and the other pharmaceutically active agent or treatment within its dosage range.

Accordingly, another embodiment provides combinations comprising an amount of at least one compound of the invention, or a pharmaceutically acceptable salt, solvate, ester or prodrug thereof, and an effective amount of one or more additional agents described above.

When the composition of the invention is prepared for oral administration, the compounds described above are generally combined with a pharmaceutically acceptable carrier, diluent or excipient to form a pharmaceutical formulation, or unit dosage form.

For oral administration, the composition may be in the form of a powder, a granular formation, a solution, a suspension, an emulsion or in a natural or synthetic polymer or resin for ingestion of the active ingredients from a chewing gum. The composition may also be presented as a bolus, electuary or paste. Orally administered compositions of the invention can also be formulated for sustained release, e.g. the compounds described above can be coated, microencapsulated, or otherwise placed within a sustained delivery device. The total active ingredients in such formulations comprise from 0.1 to 99.9% by weight of the formulation.

Thus, one or more suitable unit dosage forms comprising the compounds of the invention can be administered by a variety of routes including oral, parenteral (including subcutaneous, intravenous, intramuscular and intraperitoneal), rectal, dermal, transdermal, intrathoracic, intrapulmonary, mucosal, intraocular and intranasal (respiratory) routes. The composition may also be formulated in a lipid formulation or for sustained release, for example, using microencapsulation. The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

Pharmaceutical formulations comprising the compounds of the invention can be prepared by procedures known in the art using well-known and readily available ingredients. For example, the compound can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives.

Binding agents can also be included such as carboxymethyl cellulose, hydroxymethylcellulose, hydroxypropyl methylcellulose and other cellulose derivatives, alginates, gelatine, and polyvinylpyrrolidone. Moisturising agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the compounds of the invention can include buffering agents such as calcium carbonate, magnesium oxide and magnesium carbonate. Suitable buffering agents may also include acetic acid in a salt, citric acid in a salt, boric acid in a salt and phosphoric acid in a salt. Caplets and tablets can also include inactive ingredients such as cellulose, pregelatinised starch, silicon dioxide, hydroxyl propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatine capsules containing at least one compound of the invention can contain inactive ingredients such as gelatine, microcrystalline cellulose, sodium lauryl sulphate, starch, talc, and titanium dioxide, and the like, as well as liquid vehicles such as polyethylene glycols (PEGs) and vegetable oil. Moreover, enteric-coated caplets or tablets containing one or more compounds of the invention are designed to resist disintegration in the stomach and dissolve in the more neutral to alkaline environment of the duodenum.

The therapeutic compounds of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic compounds of the invention can also take the form of an aqueous or anhydrous solution or dispersion, or alternatively the form of an emulsion or suspension or salve.

Thus, the therapeutic compounds may be formulated for parenteral administration (e.g. by injection, for example, bolus injection or continuous infusion) and may be presented in unit dose form in ampules, pre-filled syringes, small volume infusion containers or in multi-dose containers. As noted above, preservatives can be added to help maintain the shelve life of the dosage form. The active compound(s) and other ingredients may form suspensions, solutions, or emulsions in oily or aqueous vehicles, and may contain formulatory agents such as suspending, stabilizing and/or dispersing agents. Alternatively, the active compound(s) and other ingredients may be in powder form, obtained by aseptic isolation of sterile solid or by lyophilisation from solution for constitution with a suitable vehicle, e.g., sterile, pyrogen-free water before use.

It is possible to add, if necessary, an adjuvant chosen from antioxidants, surfactants, other preservatives, film-forming, keratolytic or comedolytic agents, perfumes, flavourings and colourings. Antioxidants such as t-butylhydroquinone, butylated hydroxyanisole, butylated hydroxytoluene and a-tocopherol and its derivatives can be added.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulfoxide, glycol ethers such as the products sold under the name “Dowanol”, polyglycols and polyethylene glycols, C1-C4 alkyl esters of short-chain acids, ethyl or isopropyl lactate, fatty acid triglycerides such as the products marketed under the name “Miglyol”, isopropyl myristate, animal, mineral and vegetable oils and polysiloxanes.

Preferably, the composition is in the form of a solvent or diluent comprising one or more of the compounds as described above. Solvents or diluents may include acid solutions, dimethylsulfone, N-(2-mercaptopropionyl) glycine, 2-n-nonyl-], 3-dioxolane and ethyl alcohol. Preferably the solvent/diluent is an acidic solvent, for example, acetic acid, citric acid, boric acid, lactic acid, propionic acid, phosphoric acid, benzoic acid, butyric acid, malic acid, malonic acid, oxalic acid, succinic acid or tartaric acid.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The solvent may comprise an acetic acid solution. The solvent, for example acetic acid solution, may be present in the composition at a concentration of less than 1%, 0.5%, 0.25%, 0.1%, 0.05% or 0.01% w/w acid, for example acetic acid.

According to a yet further aspect of the invention there is provided a compound of formula I herein or a pharmaceutical composition thereto, in combination with one or more additional therapeutic agents, and a pharmaceutically acceptable adjuvant, diluent or carrier.

Non-limiting examples of additional therapeutic agents which may be useful in combination with the compounds of the present invention include those selected from the group consisting of:
(a) drugs that may be useful for the treatment of Alzheimer's disease and/or drugs that may be useful for treating one or more symptoms of Alzheimer's disease;
(b) drugs that may be useful for inhibiting the synthesis amyloid beta peptide;
(c) drugs that may be useful for treating neurodegenerative diseases; and
(d) drugs that inhibit tau aggregation or accumulation.

Non-limiting examples of additional therapeutic agents that may be useful in combination with compounds of the invention include: muscarinic antagonists (e.g., M₁ agonists (such as acetylcholine, oxotremorine or carbachol), or M₂ antagonists (such as atropine, dicycloverine, tolterodine, oxybutynin, ipratropium, methoctramine, tripitamine, or gallamine); cholinesterase inhibitors (e.g. acetyl- and/or butyrylchlolmesterase inhibitors such as donepezil (Aricept®, (±)-2,3-dihydro-5,6-dimethoxy-2-[[1-(phenylmethyl)-4-piperidinyl]methyl]-1H-inden-1-one hydrochloride), galantamine (Razadyne®), and rivastigimine (Exelon®); N-methyl-D-aspartate receptor antagonists (e.g. Namenda® (memantine HCl); combinations of cholinesterase inhibitors and N-methyl-D-aspartate receptor antagonists; gamma secretase modulators; gamma secretase inhibitors; non-steroidal anti-inflammatory agents; anti-inflammatory agents that can reduce neuroinflammation; anti-amyloid antibodies (such as bapineuzemab); vitamin E; nicotinic acetylcholine receptor agonists; CB₁ receptor inverse agonists or CB₁ receptor antagonists; antibiotics; growth hormone secretagogues; histamine H₃ antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Tau kinase inhibitors (e.g. GSK3β inhibitors, cdk5 inhibitors, or ERK inhibitors); Tau aggregation inhibitors (e.g. Rember®); RAGE inhibitors (e.g. azeliragon); anti-Abeta vaccine; APP ligands; agents that upregulate insulin, cholesterol lowering agents such as HMG-CoA reductase inhibitors (for example, statins such as atorvastatin, fluvastatin, lovastatin, mevastatin, pitavastatin, pravastatin, rosuvastatin, simvastatin) and/or cholesterol absorption inhibitors (such as ezetimibe), or combinations of HMG-CoA reductase inhibitors and cholesterol absorption inhibitors (such as, Vytorin®); fibrates (such as, clofibrate, clofibride, etofibrate, and aluminium clofibrate); combinations of fibrates and cholesterol lowering agents and/or cholesterol absorption inhibitors; nicotinic receptor agonists; niacin; combinations of niacin and cholesterol absorption inhibitors and/or cholesterol lowering agents (e.g., Simcor® (niacin/simvastatin, available from Abbott Laboratories, Inc.); LXR agonists; LRP mimics; H₃ receptor antagonists; histone deacetylase inhibitors; hsp90 inhibitors; 5-HT₄ agonists (e.g. PRX-03140 (Epix Pharmaceuticals)); 5-HT₆ receptor antagonists; mGluR₁ receptor modulators or antagonists; mGluR₅ receptor modulators or antagonists; mGluR₂₁₃ antagonists; Prostaglandin EP₂ receptor antagonists; PAI-1 inhibitors; agents that can induce Abeta efflux such as gelsolin; metal-protein attenuating compound (e.g. PBT2); and GPR³ modulators; and antihistamines such as Dimebolin (e.g. Dimebon®).

Additionally, the compounds of the present invention are well suited to formulation as sustained release dosage forms and the like. The formulations can be so constituted that they release the active compound, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

For topical administration, the active agents may be formulated as is known in the art for direct application to a target area. Forms chiefly conditioned for topical application take the form, for example, of creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic compounds of the invention can be delivered via patches or bandages for dermal administration. Alternatively, the therapeutic compounds can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 μm.

Pharmaceutical formulations for topical administration may comprise, for example, a physiologically acceptable buffered saline solution containing between about 0.001 mg/ml and about 100 mg/ml, for example between 0.1 mg/ml and 10 mg/ml, of one or more of the compounds of the present invention specific for the indication or disease to be treated.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents. The active compounds can also be delivered via iontophoresis. The percentage by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic compounds in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

The therapeutic compound may further be formulated for topical administration in the mouth or throat. For example, the active ingredients may be formulated as a lozenge further comprising a flavoured base, usually sucrose and acacia or tragacanth; pastilles comprising the composition in an inert base such as gelatine and glycerine or sucrose and acacia; and mouthwashes comprising the composition of the present invention in a suitable liquid carrier.

The compounds of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g. gelatine or blister packs from which the powder may be administered with the aid of an inhalator, insufflator, or a metered-dose inhaler.

The compounds of the present invention can also be administered in an aqueous solution when administered in an aerosol or inhaled form. Thus, other aerosol pharmaceutical formulations may comprise, for example, a physiologically acceptable buffered saline solution containing between about 0.001 mg/ml and about 100 mg/ml of one or more of the compounds of the present invention specific for the indication or disease to be treated. Dry aerosol in the form of finely divided solid particles of the compounds described above that are not dissolved or suspended in a liquid are also useful in the practice of the present invention. Compounds of the present invention may be formulated as dusting powders and comprise finely divided particles having an average particle size of between about 1 and 5 μm, alternatively between 2 and 3 μm. Finely divided particles may be prepared by pulverization and screen filtration using techniques well-known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

The compounds of the present invention can also be administered in lipid nanoparticles, for example, in a lipid matrix of all-trans retinoic acid (ATRA)-loaded nanostructured lipid carriers (NLCs), for transdermal drug delivery, such as described by Charoenputtakhun P, et al¹³.

For administration to the upper (nasal) or lower respiratory tract by inhalation, the therapeutic compounds of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Compounds of formula I in which X² is —CR¹⁰R¹¹—CR¹²R¹⁴ are either known per se or may be prepared by methods disclosed in International Patent application Nos. PCT/GB2007/003237 and PCT/GB2015/052956, which are incorporated herein by reference.

Compounds of formula I in which X² is —CR¹⁵R¹⁶, in which R¹⁵ and R¹⁶ are herein defined, may be prepared by synthetic methods known to the person skilled in the art.

The present invention will now be described by way of example only with reference to the accompanying figures in which:

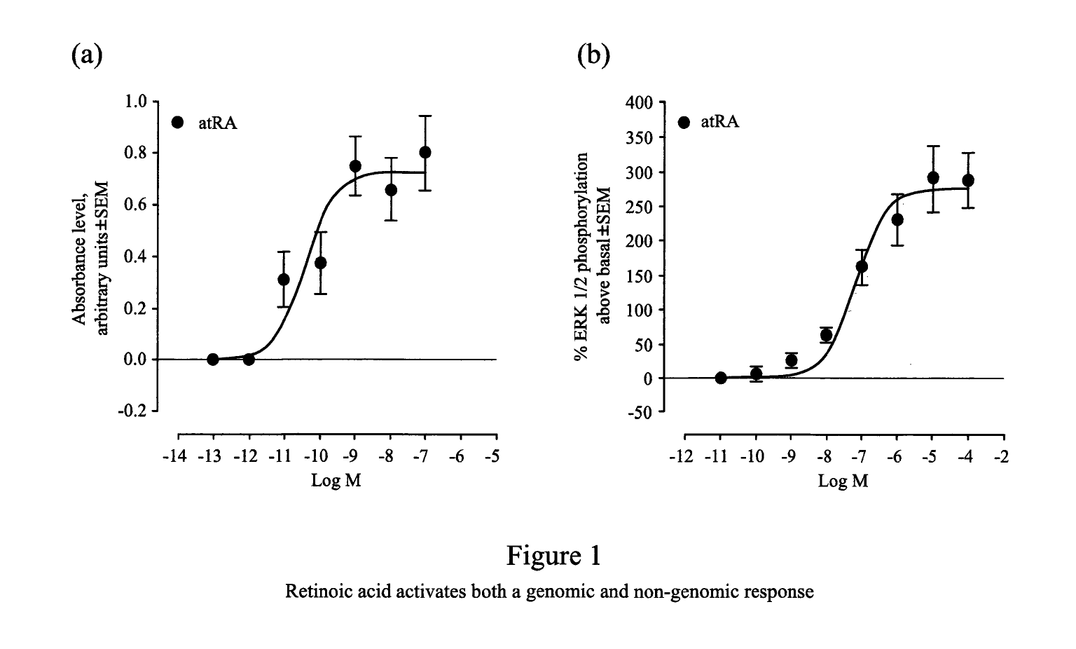

FIG. 1 illustrates both a genomic and non-genomic response with retinoic acid activation: a) Concentration Log concentration response curves for at RA-induced genomic response in RA reporter cells demonstrating its potency to induce gene expression as one aspect of the retinoids therapeutic effect. b) Concentration Log concentration response graph for ATRA-induced ERK 1/2 phosphorylation (three experiments performed in triplicate) demonstrating its efficacy to induce kinase activity.

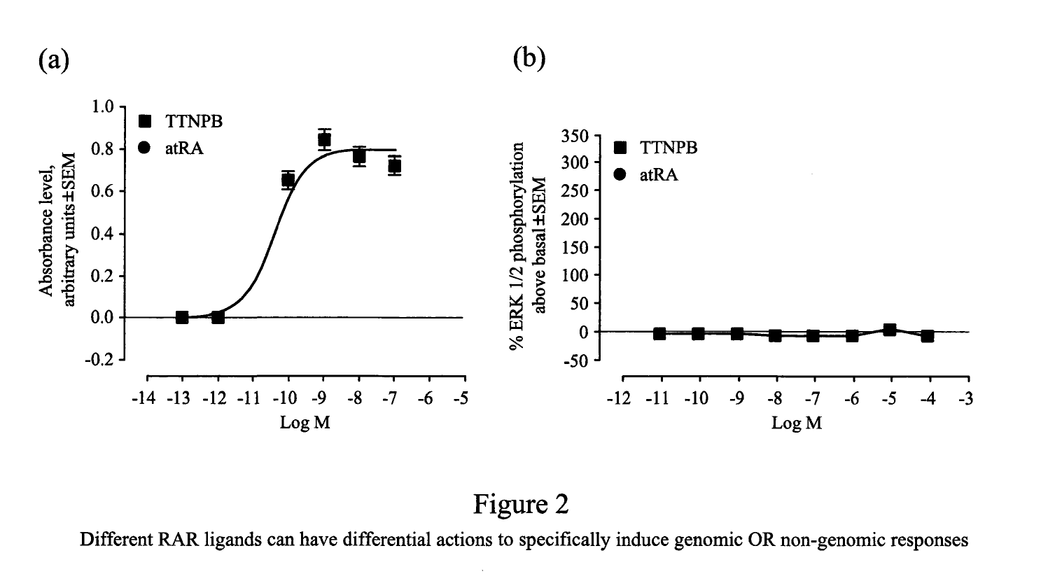

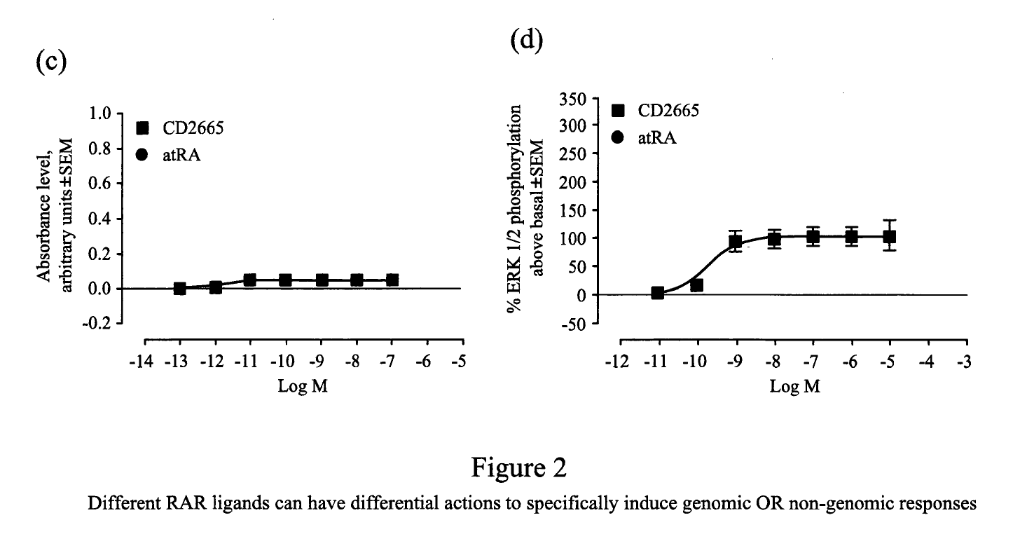

FIG. 2 illustrates how different RAR ligands can have differential actions to specifically induce genomic or non-genomic responses: (a) Concentration response graph for log (agonist) vs. response linear regression in evaluating TTNPB induced genomic response with Sil-15 reporter cells (three experiments performed in triplicate). (b) Concentration response graph for log (agonist) vs. response linear regression in evaluating TTNPB-induced ERK 1/2 phosphorylation (three experiments performed in triplicate). (c) Concentration response graph for log (agonist) vs. response linear regression in evaluating CD2665 induced genomic response with Sil-15 reporter cells (three experiments performed in triplicate). (d) Concentration response graph for log (agonist) vs. response linear regression in evaluating CD2665-induced ERK 1/2 phosphorylation.

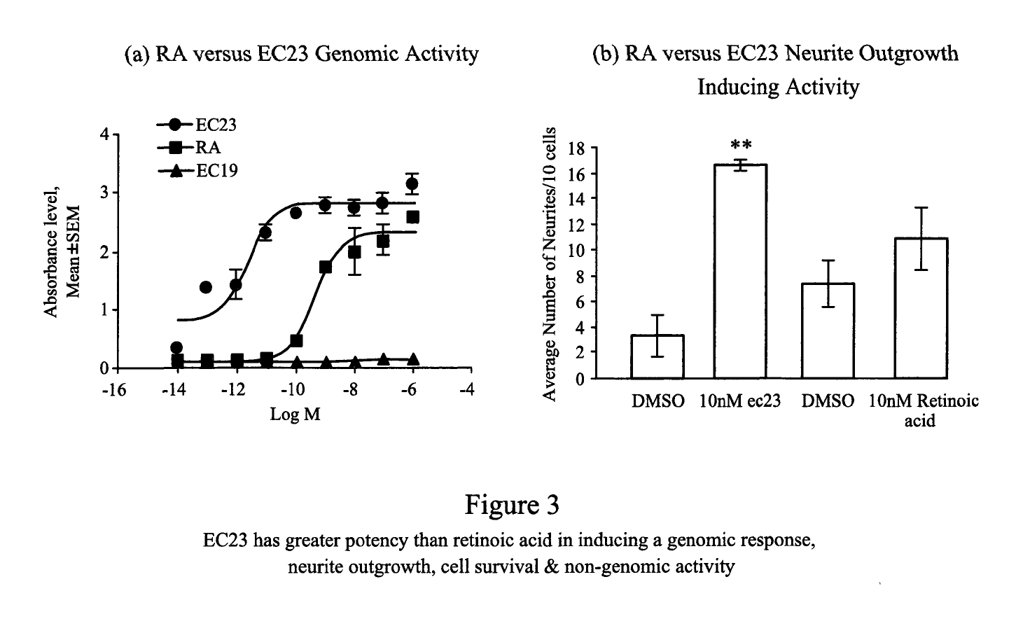

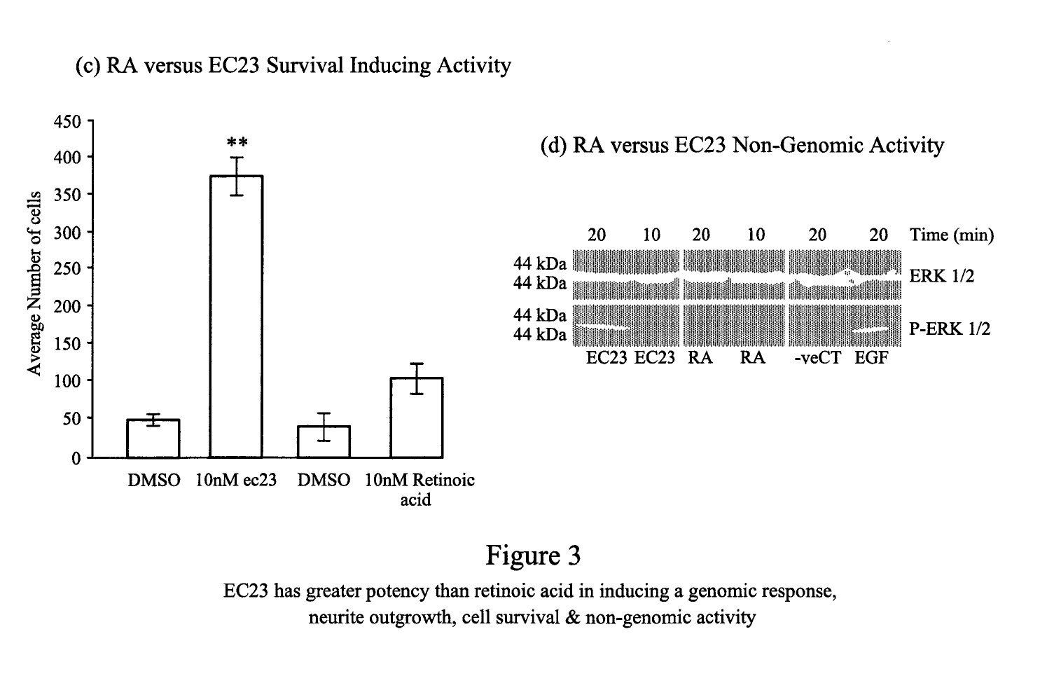

FIG. 3 illustrates that compound EC23 has a greater potency than retinoic acid in inducing a genomic response, neurite outgrowth, cell survival and non-genomic activity: (a) The concentration response graph for induction of a RAR driven reporter gene by EC23® and the RXR specific ligand EC19,^{ref 3} showing the high potency of EC23® as a RAR transactivator compared to retinoic acid. (b) The influence of a low concentration of retinoids on number of neurites from SH-SS5Y cells was determined. Whereas 10 nM of retinoic acid had no significant effect on neurite number the same concentration of EC23® had a strong and significant effect on number of neurites compared to control (**p<0.001). (c) A low concentration of EC23® (10 nM) greatly enhances the number of SH-SY5Y cells compared to the same concentration of retinoic acid (**p<0.001). (d) Using western blotting of phosphorylated ERK as a measure of non-genomic activation then 10 μM EC23® is much greater in its capacity to induce ERK phosphorylation than the same concentration of retinoic acid after 20 minutes (lower bands) and was similar to the very potent protein activator epidermal growth factor (EGF).

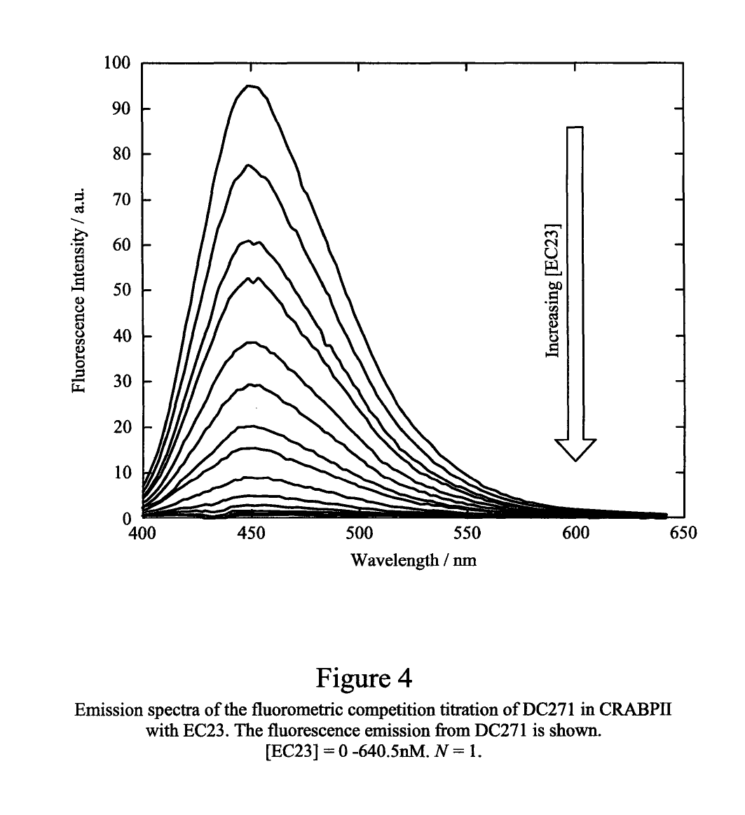

FIG. 4 illustrates emission spectra of the fluorometric competition titration of DC271 in CRABPII with EC23®. The fluorescence emission from DC271 is shown. [EC23]=0−640.5 nM. N=1.

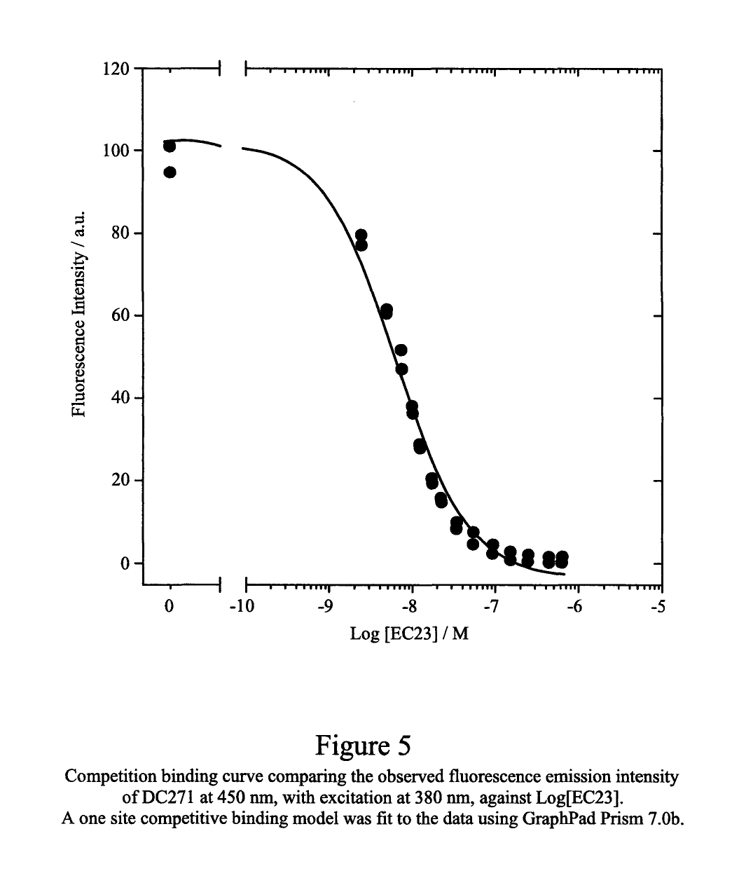

FIG. 5 illustrates a competition binding curve comparing the observed fluorescence emission intensity of DC271 at 450 nm, with excitation at 380 nm, against Log[EC23®]. A one site competitive binding model was fit to the data using GraphPad Prism 7.0b to calculate the Kᵢ of the competition of EC23 with DC271 bound to CRABPII.

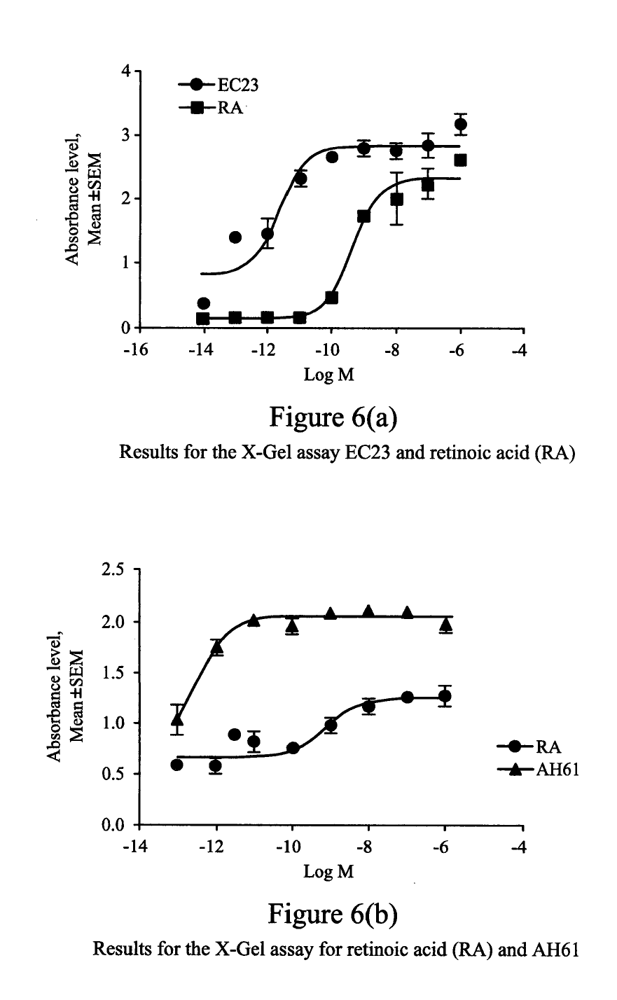

FIGS. 6(a) and (b) illustrate the results for the X-Gal assay of (a) EC23 and retinoic acid (RA); and (b) retinoic acid (RA) and AH61 ((2E,4E)-2-Methyl-7-(5,5,8,8-tetramethyl-5,6,7,8-tetrahydronaphthalen-2-yl)-hepta-2,4-dien-6-ynoic acid).

### BIOLOGICAL EVALUATION

1. CNS Function of Retinoic Acid

The action of retinoic acid in the brain, the relative importance of genomic versus non-genomic activity of retinoic acid was investigated by determining whether some RAR ligands preferentially activated one pathway. Retinoic acid (FIG. 1) was used as a compound known to induce both genomic and non-genomic pathways of retinoic acid signalling. We measured its relative activity as an inducer of gene transcription using a construct in which the promoter element to which the retinoic acid receptor binds (retinoic acid response element, RARE) drives a LacZ reporter (genomic response). This was compared to a cell based assay to measure induction of ERK 1/2 phosphorylation (non-genomic response). ATRA was found to potently induce genomic activation (FIG. 1a) with a mean EC₅₀ value of 188.4 pM with 95% confidence interval from 90.7 pM to 391.4 pM (pEC50±S.E.M. and efficacy (Eₘₐₓ) with 95% confidence intervals. ATRA was also a strong activator of ERK 1/2 kinase phosphorylation (FIG. 1b) with mean EC₅₀ value of 67.5 nM with 95% confidence interval from 32.8 nM to 138.8 nM.

2. Genomic and Non-Genomic Activities of Ligands

A study of ten commercially-available synthetic ligands found a previously unknown variance in their relative genomic and non-genomic activities, and little correlation between induction of these, suggesting that quite different pathways mediate these two activities. For instance, our results show that the RAR-α, -β and -γ ligand, TTNPB, is an extremely potent analogue of retinoic acid, can potently activate transcription via a RARE (FIG. 2a), but does not activate kinase activity (FIG. 2b) while the selective RAR-β and -γ receptor ligand, CD2665, shows no sign of activity to regulate gene expression via a RARE (FIG. 2c) but has significant kinase activity (FIG. 2d).

Analysis of these results reveals that commercially-available RAR ligands can be quite different in their genomic versus non-genomic activity. Initial analysis of ten commercial RAR ligands (results not shown), suggests that high neurotrophic activity requires BOTH genomic AND non-genomic activity.

3. Retinoic Acid in Neuroplasticity and Neurite Outgrowth

The capacity of retinoic acid to promote a second feature of neuroplasticity, neurite outgrowth, is shown in preliminary results using the SH-SY5Y cell line in a screening assay. Retinoic acid induces gene transcription of a RAR driven reporter gene (FIG. 3a) and promotes neurite outgrowth (FIG. 3b), while the increase in cell number promoted by retinoic acid (FIG. 3c) is indicative of its neurotrophic activity and support of cell survival.

Greatly superseding retinoic acid in these functions is the compound EC23® which has up to 500-fold greater activity than retinoic acid (ATRA) in its capacity to induce transactivation of a RAR driven reporter gene (FIG. 3a). Furthermore, the ligand is more potent than ATRA in its ability to promote cell survival as well as neurite outgrowth, indicative of its ability to act as a neurotrophic factor. EC23® is photostable and, unlike retinoic acid, does not induce its own catabolism in vivo.

4. CRABPII Assay

Assay Summary

A number of highly fluorescent analogues of retinoic acid (ATRA) have been synthesised at Durham University for use in a variety of imaging and biophysical studies. The fluorescence emission of these fluorescent retinoids, including DC271, is highly solvatochromatic and the intensity of this emission is strongly dependent on environment. Therefore, in a nonpolar solvent, the emission is blue shifted (425-500 nm) and intense, while in a polar solvent the emission is red shifted (500-600 nm) and weak (severely quenched in water).

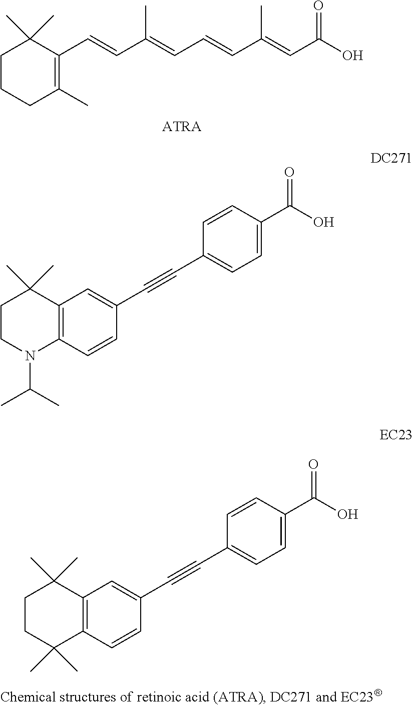

ATRA is known to bind strongly to a number of retinoid-binding proteins around the human body, such as Cellular Retinoic Acid Binding Protein II (CRABPII), and each of these proteins possesses a very hydrophobic binding pocket. Fluorescent retinoid, DC271 also binds to CRABPII, and exhibits intense fluorescence emission intensity when bound. When EC23® is titrated against CRABPII-DC271 complex the reduction in fluorescence emission intensity can be used to produce a competition binding curve allowing the estimation of the Kᵢ (inhibition constant) of the competition of EC23® for DC271 bound to CRABPII.

Competition Assay Results

DC271 exhibits a dissociation constant against of CRABPII of K_{d}=49.1 ±2.6 nM. For the competition with EC23® a solution of [DC271] =49.8 nM and [CRABPII]=75 nM was prepared in PBS buffer (20 mM K₂HPO₄, 100mM KCl, pH 7.4 buffer) and the fluorescence intensity was measured at 450 nm with excitation at 380 nm. Aliquots of EC23® (dissolved in EtOH) were added to DC271-CRABPII complex until the fluorescence emission intensity reached a minimum value, indicating that EC23 had outcompeted all of the DC271 in solution (FIG. 4). The emission intensity of DC271 was plotted against Log[EC23], and a one site competitive binding model was fit to the resultant data (FIG. 5). This provided an estimation of the Log Kᵢ of the competitive binding reaction (Table 1). This indicated that, as predicted, EC23® exhibits a strong binding affinity for CRABPII and, therefore, can be defined as a retinoid.

TABLE 1
Log Kᵢ and Kᵢ values determined from the fluorometric competitive
binding titration of DC271 in CRABPII with EC23 ®.

| Retinoid | Log Kᵢ/M ± S.E. | Kᵢ/nM |
|---|---|---|
| EC23 ® | −8.50 ± 0.04 | 3.16 |

5. X-Gal Assay

Sil-15 reporter cells (also called F9-RARE-lacZ cells) were used in the X-Gal Assay. This cell line was derived from a stable transfection of F9 teratocarcinoma cells with a plasmid containing the LacZ gene under the control of a promoter linked to a retinoic acid response element (RARE). These cells were used to visually detect and semi-quantify the transcriptional activity of retinoic acid and other retinoids by monitoring β-galactosidase activity produced by the reporter cells in response to the retinoic acid/retinoids in the surrounding medium.

Sil-15 cells were plated in a 0.1% gelatin-coated 96-well plate and grown to about 85-90% confluence in Dulbecco's modified Eagle's medium (DMEM) containing 10% foetal calf serum and 0.8 mg/ml G418 sulfate for selection.

Serial dilutions of retinoids were prepared from the stock solutions under a red/orange light. A standard curve of RA was included in the experiment as a positive control. The RA/chemicals dilutions were added above the seeded Sil-15 cells in the 96-well plate and then the plate was wrapped with aluminium foil and incubated overnight in a humid atmosphere containing 5% CO₂ at 37° C. All the concentrations for the RA and the two chemicals were tested in triplicates.

The next day, Sil-15 cells were washed twice with 1× PBS, fixed with 100 μl per well of 1% glutaraldehyde fixation solution for 15 minutes, washed again two times with 1× PBS and finally β-galactosidase activity was visualized by adding 100 μl of a freshly prepared X-Gal developing solution (5-bromo-4-chloro-3-indolyl-β-d-galactopyranoside) to each well. The plate was incubated overnight at 37° C. in 5% CO₂. The plate was read on an Emax microplate reader at 650 nm.

### REFERENCES

1. Tang, X. H. & Gudas, L. J. Retinoids, retinoic acid receptors, and cancer. Annual review of pathology 6, 345-364 (2011).
2. Theodosiou, M., Laudet, V. & Schubert, M. From carrot to clinic: an overview of the retinoic acid signaling pathway. Cellular and molecular life sciences: CMLS 67, 1423-1445 (2010).
3. Connor, M. J. & Sidell, N. Retinoic acid synthesis in normal and Alzheimer diseased brain and human neural cells. Mol Chem Neuropathol 30, 239-252. (1997).
4. Mingaud, F., et al. Retinoid hyposignaling contributes to aging-related decline in hippocampal function in short-term/working memory organization and long-term declarative memory encoding in mice. J Neurosci 28, 279-291 (2008).
5. Touyarot, K., et al. A mid-life vitamin A supplementation prevents age-related spatial memory deficits and hippocampal neurogenesis alterations through CRABP-I. PloS one 8, e72101 (2013).
6. Bourdel-Marchasson, I., et al. Antioxidant defences and oxidative stress markers in erythrocytes and plasma from normally nourished elderly Alzheimer patients. Age Ageing 30, 235-241 (2001).
7. Rinaldi, P., et al. Plasma antioxidants are similarly depleted in mild cognitive impairment and in Alzheimer's disease. Neurobiol Aging 24, 915-919 (2003).
8. Shearer, K. D., Stoney, P. N., Morgan, P. J. & McCaffery, P. J. A vitamin for the brain. Trends in neurosciences 35, 733-741 (2012).
9. Goncalves, M. B., et al. Amyloid beta inhibits retinoic acid synthesis exacerbating Alzheimer disease pathology which can be attenuated by an retinoic acid receptor alpha agonist. The European journal of neuroscience 37, 1182-1192 (2013).
10. Ono, K., et al. Vitamin A exhibits potent antiamyloidogenic and fibril-destabilizing effects in vitro. Experimental neurology 189, 380-392 (2004).
11. Sahin, M., Karauzum, S.B., Perry, G., Smith, M.A. & Aliciguzel, Y. Retinoic acid isomers protect hippocampal neurons from amyloid-beta induced neurodegeneration. Neurotox Res 7, 243-250 (2005).
12. Sodhi, R. K. & Singh, N. Liver X receptors: emerging therapeutic targets for Alzheimer's disease. Pharmacological research: the official journal of the Italian Pharmacological Society 72, 45-51 (2013).
13. Charoenputtakhun P, et al in Pharm Dev Technol. 2014 March; 19(2):164-72. doi: 10.3109/10837450.2013.763261. Epub 2013 Jan. 28

1. A compound of formula 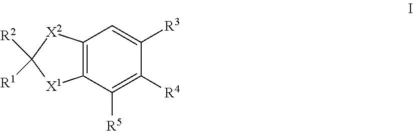
in which
X¹ is —NCHR⁶R⁷, —CR⁸R⁹;
X² is —CR¹⁰R¹¹—CR¹²R¹⁴ or —CR¹⁵R¹⁶;
R¹, R², R¹⁰ and R¹¹, which may be the same or different, are each hydrogen or alkyl C1-4, or together one pair of R¹ and R¹⁰ or R² and R¹¹ represent a 5- or 6-membered ring, or together one pair of R¹ and R¹⁰ or R² and R¹¹ represent a bond;
R⁴ and R⁵ are each hydrogen;
R⁶, R⁷, R⁸ and R⁹, which may be the same or different, are each hydrogen, alkyl C1-10 acyl;
R¹² and R¹⁴, which may be the same or different, are each hydrogen, alkyl C1-4; or together one pair of R¹⁰ and R¹² or R¹¹ and R¹⁴ represent a bond, or R¹² and R¹⁴ together form a group:
═CR¹⁷R¹⁸
provided that the pair of R¹⁰ and R¹² or R¹¹ and R¹⁴ does not represent a bond if a pair from R¹, R², R¹⁰ and R¹¹ represents a bond;
R¹⁵ and R¹⁶, which may be the same or different, are each hydrogen or alkyl C1-4, or together one pair of R¹ and R¹⁵ or R² and R¹⁶ represent a 5- or 6-membered ring;
R¹⁷ and R¹⁸, which may be the same or different, are each hydrogen or alkyl C1-10; and
R³ is a group II, III, IV, VI, VII, X, XI or XII: 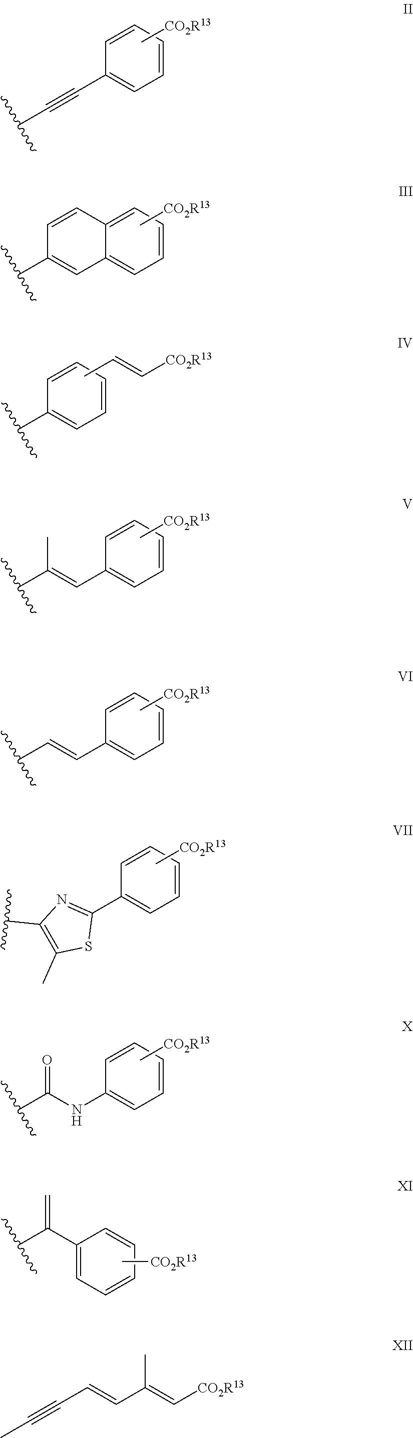
in which R¹³ is hydrogen, alkyl C1-10 or haloalkyl C1-10;
and isomers thereof,
in free or in salt form;
for use in the treatment or alleviation of an RAR mediated condition.

2. The compound of formula I according to claim 1 wherein R³ is a group II, III or IV.

3-6. (canceled)

7. The compound of formula I according to claim 1 wherein R¹, R², R¹⁰, R¹¹, R¹², R¹⁴, R¹⁵ and R¹⁶ are each hydrogen.

8. The compound of formula I according to claim 1 wherein one pair of R¹ and R¹⁰ or R² and R¹¹ represent a bond.

9. The compound of formula I according to claim 1 which is a compound of formula XIII: 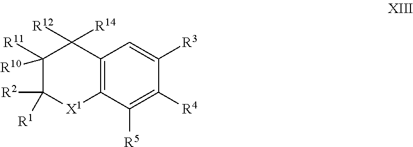
in which
X¹, R¹, R², R³, R⁴, R⁵, R¹⁰, R¹¹, R¹² and R¹⁴, are each as defined in claim 1.

10. The compound of formula XIII according to claim 2 in which X¹ is —CR⁸R⁹; and
R⁸ and R⁹ are each as defined in claim 9.

11. The compound of formula XIII according to claim 2 in which X¹—NCHR⁶R⁷; and
R⁶ and R⁷ are each as defined in claim 9.

12. The compound of formula I according to claim 1 which is a compound of formula XIV: 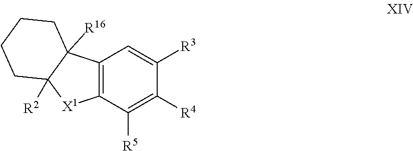
in which
X¹, R², R³, R⁴, R⁵ and R¹⁶ are each as defined in claim 1.

13-14. (canceled)

15. The compound according to claim 1 which is selected from the group consisting of:
4-(5,5,8,8-tetramethyl-5,6,7,8-tetrahydro-naphthalen-2-ylethynyl) benzoic acid;
4-2-[4,4-dimethyl-1-(propan-2-yl)-1,2,3,4-tetrahydroquinolin-6-yl]ethynylbenzoic acid;
6-(1,4,4-trimethyl-1,2,3,4-tetrahydroquinolin-6-yl)-naphthalene-2-carboxylic acid methyl ester;
3-[4-(1,4,4-trimethyl-1,2,3,4-tetrahydroquinolin-6-yl)-phenyl]-acrylic acid methyl ester; and
4-2-[2,4,4-trimethyl-1-(propan-2-yl)-1,4-dihydroquinolin-6-yl]ethynylbenzoic acid;
in free or in salt form;
for use in the treatment or alleviation of an RAR mediated condition.

16. The compound according to claim 1 wherein the RAR mediated condition is a neurodegenerd ondition selected from one or more of, multiple sclerosis, Parkinson's disease, stroke, traumatic brain injury, epilepsy, cognitive disorders, memory impairment, memory deficit, senile dementia, Alzheimer's disease, early stage Alzheimer's disease, intermediate stage Alzheimer's disease, late stage Alzheimer's disease, cognitive impairment, mild cognitive impairment and ALS (amyotrophic lateral sclerosis

17-18. (canceled)

19. The use of a compound according to claim 1 in the manufacture of a medicament for use in the treatment or alleviation of an RAR mediated condition.

20. The use of a compound according to claim 19 wherein the RAR mediated condition is a neurodegenerative condition selected from one or more of, multiple sclerosis, Parkinson's disease, stroke, traumatic brain injury, epilepsy, cognitive disorders, memory impairment, memory deficit, senile dementia, Alzheimer's disease, early stage Alzheimer's disease, intermediate stage Alzheimer's disease, late stage Alzheimer's disease, cognitive impairment, mild cognitive impairment and ALS (amyotrophic lateral sclerosis).

21-22. (canceled)

23. A method of treatment of a patient suffering an RAR mediated condition, said method comprising administering to a patient a therapeutically effective amount of a compound of formula I: 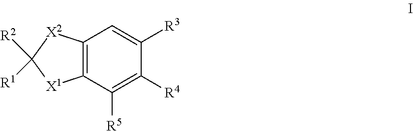
in which
X¹ is —NCHR⁶R⁷, —CR⁸R⁹;
X² is —CR¹⁰ R¹¹—CR¹²R¹⁴ or —CR¹⁵R¹⁶;
R¹, R², R¹⁰ and R¹¹, which may be the same or different, are each hydrogen or alkyl C1-4, or together one pair of R.¹ and R¹⁰ or R² and R¹¹ represent a 5- or 6-membered ring, or together one pair of R¹ and R¹⁰ or R² and R¹¹ represent a bond;
R¹ and R⁵ are each hydrogen;
R⁶, R⁷, R⁸ and R⁹, which may be the same or different, are each hydrogen, alkyl C1-10 acyl;
R¹² and R¹⁴, which may be the same or different, are each hydrogen, alkyl C1-4; or together one pair of R¹⁰ and R¹² or R¹¹ and R¹⁴ represent a bond, or R¹² and R¹⁴ together form a group:
═CR¹⁷R¹⁸
provided that the pair of R¹⁰ and R¹² or R¹¹ and R¹⁴ does not represent a bond if a pair from R¹, R², R¹⁰ and R¹¹ represents a bond;
R¹⁵ and R¹⁶, which may be the same or different, are each hydrogen or alkyl C1-4, or together one pair of R¹ and R¹⁵ or R² and R¹⁶ represent a 5- or 6-membered ring;
R¹⁷ and R¹⁸, which may be the same or different, are each hydrogen or alkyl C1-10; and
R³ is a group II, III, IV, V, VI, VII, X, XI or XII: 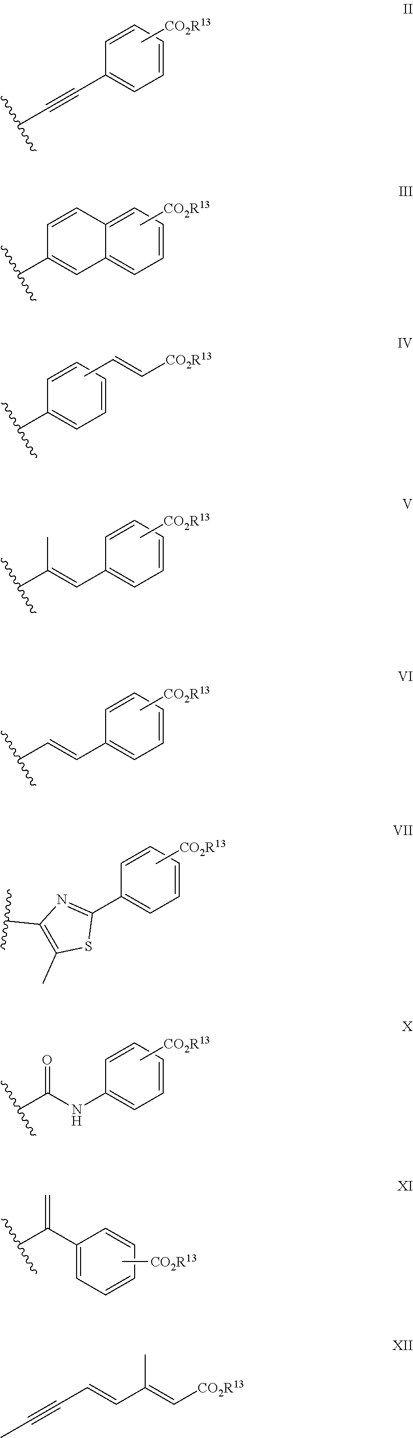
in which R¹³ is hydrogen, alkyl C1-10 or haloalkyl C1-10;
and isomers thereof;
in free or in salt form.

24. The method according to claim 23 wherein R³ is a group II, III or IV.

25-28. (canceled)

29. The method according to claim 23 wherein R¹, R², R¹⁰, R¹¹, R¹², R¹⁴, R¹⁵ and R¹⁶ are each hydrogen.

30. The method according to claim 23 wherein one pair of R¹ and R¹⁰ or R² and R¹¹ represent a bond.

31. The method according to claim 23 which is a compound of formula XIII: 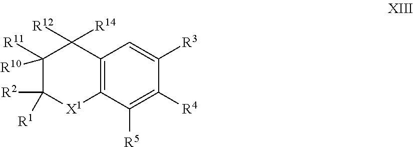
in which
X¹, R¹, R², R³, R⁴, R⁵, R¹⁰, R¹¹, R¹² and R¹⁴ are each as defined in claim 1

32-33. (canceled)

34. The method according to claim 23 which is a compound of formula XIV: 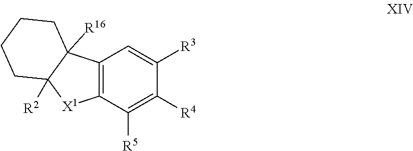
in which
X¹, R², R³, R⁴, R⁵ and R¹⁶ are each as defined in claim 23.

35-36. (canceled)

37. The method according to claim 24 wherein the compound of formula I is selected from the group consisting of:
4-(5,5,8,8-tetramethyl-5,6,7,8-tetrahydro-naphthalen-2-ylethynyl) benzoic acid;
4-2-[4,4-dimethyl-1-(propan-2-yl)-1,2,3,4-tetrahydroquinolin-6-yl]ethynylbenzoic acid;
6-(1,4,4-trimethyl-1,2,3,4-tetrahydroquinolin-6-yl)-naphthalene-2-carboxylic acid methyl ester;
3-[4-(1,4,4-trimethyl-1,2,3,4-tetrahydroquinolin-6-yl)-phenyl]-acrylic acid methyl ester; and
4-2-[2,4,4-trimethyl-1-(propa.n-2-yl)-1,4-dihydroquinolin-6-yl]ethynylbenzoic acid;
in free or in salt form.

38. The method of treatment according to claim 23 wherein the RAR mediated. condition is a neurodegenerative condition selected from one or more of, multiple sclerosis, Parkinson's disease, stroke, traumatic brain injury, epilepsy, cognitive disorders, memory impairment, memory deficit, senile dementia, Alzheimer's disease, early stage Alzheimer's disease, intermediate stage Alzheimer's disease, late stage Alzheimer's disease, cognitive impairment, mild cognitive impairment and ALS(amyotrophic lateral sclerosis).

39-40. (canceled)

41. The pharmaceutical composition comprising a compound of formula I according to claim 1, and a pharmaceutically acceptable adjuvant, diluent or carrier, for use in the treatment or alleviation of an RAR mediated condition as herein defined.

42. The method of preparing a pharmaceutical composition according to claim 41, said method comprising the step of admixing a compound of formula I and a pharmaceutically acceptable adjuvant, diluent or carrier.

43. A compound of formula I: 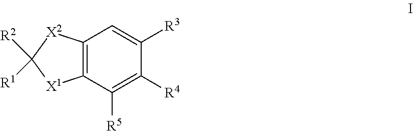
in which
X² is —CR¹⁵R¹⁶; and
X¹, R¹, R², R³, R⁴, R⁵, R¹⁵ and R¹⁶ are each as herein defined;
and isomers thereof;
in free or in salt form.

44. The compound of formula I according to claim 43 compound of formula XIV: 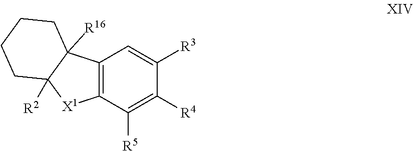
in which
X¹, R², R³, R⁴, R⁵ and R¹⁶ are each as defined in claim 43;
and isomers thereof;
in free or in salt form.

45. A compound of formula XIV: 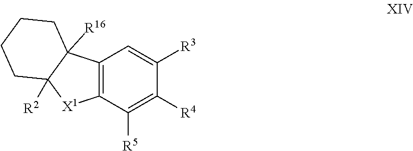
in which
X¹ is —NCHR⁶R⁷; and
R², R³, R⁴, R⁵, R⁶, R⁷ and R¹⁶ are each as defined in claim 43;
and isomers thereof,
in free or in salt form.

46. A compound of formula XIV: 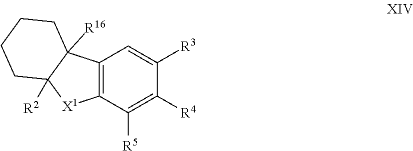
X¹ is —CR⁸R⁹; and
R², R³, R⁴, R⁵, R⁸, R⁹ and R¹⁶ are each as defined in claim 43;
and isomers thereof,
in free or in salt form.

47-48. (canceled)
